(19) United States
(12) Patent Application Publication
(10) Pub. No.: US 2025/0166823 A1
(43) Pub. Date: May 22, 2025

(54) INFORMATION PROCESSING DEVICE, INFORMATION PROCESSING SYSTEM, INFORMATION PROCESSING METHOD AND PROGRAM

(71) Applicant: MITSUBISHI TANABE PHARMA CORPORATION, Osaka (JP)

(72) Inventor: Satoshi IWASAKI, Jersey City, NJ (US)

(73) Assignee: MITSUBISHI TANABE PHARMA CORPORATION, Osaka (JP)

(21) Appl. No.: 18/839,045

(22) PCT Filed: Feb. 23, 2023
(86) PCT No.: PCT/US2023/013664

§ 371 Date: Aug. 16, 2024

Related U.S. Application Data
(60) Provisional application No. 63/313,084, filed on Feb. 23, 2022.

(51) Int. Cl. G16H 50/20; G16H 10/20; G16H 10/60; G16H 80/00

(52) U.S. Cl. CPC G16H 50/20; G16H 10/20; G16H 10/60; G16H 80/00

(57) ABSTRACT

An information processing device includes a data acquisition part that acquires one or more neuromuscular disease-related user data selected from (a)-(k) multiple times in a predetermined period; and an information generation part that generates to-be-provided information to be provided to a predetermined terminal based on the user data. (a) Typing operation-related data, (b) walking-related data, (c) utterance-related data, (d) sleep-related data, (e) breathing-related data, (f) facial expression-related data, (g) fine motor movement-related data, (h) gross motor movement-related data, (i) questionnaire answers regarding disease symptoms, (j) information automatically collected with built-in sensors of devices, and (k) data from medical institutions.

### CROSS-REFERENCE TO RELATED APPLICATIONS

The present application claims the benefit of priority to U.S. Application No. 63/313,084, filed Feb. 23, 2022. The entire contents of this application are incorporated herein by reference.

### BACKGROUND OF THE INVENTION

### Field of the Invention

The present invention relates to an information processing device, an information processing system, an information processing method and a program.

### Description of Background Art

Neuromuscular disease is a general term for diseases that cause movement disorders due to a lesion of the nerve itself such as the brain, spinal cord, and peripheral nerves, or a lesion of the muscle itself, and examples of typical diseases include Parkinson's disease, spinocerebellar degeneration, amyotrophic lateral sclerosis, neuritis and myelitis caused by viruses or fungi, myasthenia gravis, muscular dystrophy, and polymyositis (see Intractable Disease Information Center Internet <URL: https://www.nanbyou.or.jp/entry/5347#01>, Japanese Society of Neurology Internet <URL: https://www.neurology-jp.org/public/disease/index.html#about>, Japanese Society of Orthopedic Surgery, List of Symptoms of Neuromuscular Diseases Internet <URL: https://www.joa.or.jp/public/sick/body/nerve.html>). Movement disorder is a common main symptom of these neuromuscular diseases.

Among neuromuscular diseases, for example, amyotrophic lateral sclerosis (hereinafter also referred to as “ALS”) is a rapidly progressive fatal and serious disease in which voluntary movements are impaired due to selective degenerative loss of upper and lower motor neurons, weakness of upper and lower limbs or bulbar palsy and respiratory muscle palsy gradually progress, and respiratory management is often required due to respiratory failure 2-5 years after the onset of the disease.

There are individual differences in type and progression of atrophied muscles in ALS patients. Further, since ALS does not have a specific marker, the current diagnosis is basically an exclusion diagnosis. As diagnostic criteria, for example, there are revised El Escorial diagnostic criteria. However, diagnostic sensitivity is low, and in practice, clinical diagnosis is comprehensively performed. There is also an electrophysiological test. However, since this test imposes a heavy burden on patients and cannot track progression or severity of a pathological condition, progression of ALS is visually evaluated and highly sensitive diagnosis at an early stage is difficult. Therefore, in order to quantitatively and objectively evaluate limb ability, for example, there is a technology that improves convenience in measuring limb ability using a motion capture technology (for example, Japanese Patent No. 6465419 and the like). The entire contents of these publications are incorporated herein by reference.

### SUMMARY OF THE INVENTION

According to one aspect of the present invention, an information processing device includes a data acquisition part that acquires one or more neuromuscular disease-related user data selected from the following (a)-(k) multiple times in a predetermined period, and an information generation part that generates to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

According to another aspect of the present invention, an information processing device includes a data acquisition part that acquires one or more direct or indirect motor nervous system dysfunction-related user data selected from the following (a)-(k) multiple times in a predetermined period, and an information generation part that generates to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

According to yet another aspect of the present invention, an information processing system includes a data acquisition part that acquires one or more neuromuscular disease-related user data selected from the following (a)-(k) multiple times in a predetermined period, and an information generation part that generates to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

According to still another aspect of the present invention, an information processing method, in which a computer is used, includes acquiring one or more neuromuscular disease-related user data selected from the following (a)-(k) multiple times in a predetermined period, and generating to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

Still another aspect of the present invention is a program for causing a computer to execute an information processing method. The program causes the computer to execute as the information processing method including acquiring one or more neuromuscular disease-related user data selected from the following (a)-(k) multiple times in a predetermined period, and generating to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 illustrates an example of a structure of an information processing system that includes an information processing device;

FIG. 2 illustrates an example of a hardware structure of the information processing device;

FIG. 3 illustrates an example of a software structure of the information processing device;

FIG. 4 illustrates an example of a structure of user basic information stored in a user information storage part;

FIG. 5 illustrates an example of a structure of user data stored in a user data storage part;

FIG. 6 illustrates an example of a structure of doctor input information stored in a doctor input information storage part;

FIG. 7 illustrates an example of a structure of to-be-provided information stored in a to-be-provided information storage part; and

FIG. 8 illustrates a flow of processing executed in the information processing device.

### DETAILED DESCRIPTION OF THE EMBODIMENTS

Embodiments will now be described with reference to the accompanying drawings, wherein like reference numerals designate corresponding or identical elements throughout the various drawings.

A specific example of an information processing device 10 according to an embodiment of the present invention is described with reference to the drawings.

In the present embodiment, examples of “neuromuscular diseases” include Parkinson's disease, Huntington's disease, amyotrophic lateral sclerosis, spinocerebellar degeneration, progressive supranuclear palsy, multiple system atrophy, multiple sclerosis, neuromyelitis optica, adrenoleukodystrophy, metamorphic white matter dystrophy, hyperglutaric acidemia type I, phenylketonuria, GM1 gangliosidosis, GM2 gangliosidosis, mucolipidosis type II (I-cell disease), Angelman syndrome, Krabbe disease, Batten disease, mucopolysaccharidosis, Rett's disease, Niemann-Pick disease A, B, C, spinal cord injury, inclusion body myositis, myasthenia gravis, hereditary spasm and paralysis, primary lateral sclerosis, Charcot-Marie-Tooth disease, spinal muscular atrophy, Friedreich's ataxia, dermatomyositis, polymyositis, Guillain-Barré Syndrome, chronic inflammatory, demyelinating polyneuritis, Lumber Eaton myasthenia gravis, multifocal motor neuropathy, anti-MAG peripheral neuropathy, facial scapulohumeral muscular dystrophy, muscular dystrophy, myotonic dystrophy, Duchenne muscular dystrophy, facial scapulohumeral muscular dystrophy, spinal and bulbar muscular atrophy, mitochondrial disease, Leigh encephalopathy, MELAS (mitochondrial encephalomyopathy, lactic acidosis, stroke-like seizure syndrome), fragile X-associated tremor/ataxia syndrome (FXTAS), Periceus-Merzbach's disease (PMD), neuritis and myelitis caused by viruses and fungi, cerebral infarction, cervical spondylosis, spondylosis, and the like. Cerebral infarction, cervical spondylosis, and myelopathy are diseases that cause movement disorders, although they are not generally included in neuromuscular diseases, and are synonymously regarded as diseases included in neuromuscular diseases in the present embodiment.

In the present embodiment, a “direct or indirect motor nervous system dysfunction” refers to, for example, a motor nervous system dysfunction among symptoms or test results of a patient that a doctor confirms with the patient in order to make a diagnosis of a neuromuscular disease. Examples of symptoms or test results that are directly related to a motor nervous system dysfunction include those related to typing, walking, utterance, breathing, facial expression, or fine or gross motor movements. Further, examples of symptoms or test results that are indirectly related to a motor nervous system dysfunction include those related to sleep.

### Structure

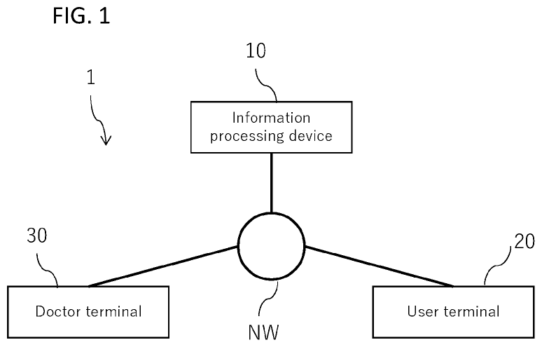

FIG. 1 illustrates an example of an information processing system 1 that includes the information processing device 10 according to the present embodiment. In this embodiment, the information processing device 10 is communicably connected via a network (NW) to a user terminal 20 used by a user such as a patient and a doctor terminal 30 used by a doctor.

In present embodiment, the network (NW) is, for example, the Internet. The network (NW) is constructed by, for example, a public telephone line network, a mobile phone line network, a wireless communication network, an Ethernet (registered trademark), and the like.

The information processing device 10 is a terminal managed by a medical institution or an organization that provides medical information, and forms a part of an information processing system 1 by executing information processing with the user terminal 20 and the doctor terminal 30 via the network (NW). The information processing device 10 may be, for example, a general-purpose computer such as a workstation or a personal computer, or may be logically realized by cloud computing. In such an information processing device 10, an application or the like capable of communicating with the user terminal 20 and the doctor terminal 30 may be installed, or a browser for accessing a web service that enables the communication may be installed.

The user terminal 20 is a terminal that is mainly used by a user to input data or the like, and executes information processing with the information processing device 10 and the doctor terminal 30 via the network (NW). The user terminal 20 may be, for example, a general-purpose computer such as a workstation or a personal computer, or a portable communication device or the like such as a smartphone. Further, the user terminal 20 may be a digital device such as a wearable device that the user can wear. In such a user terminal 20, an application or the like capable of communicating with the information processing device 10 or the doctor terminal 30 may be installed, or a browser for accessing a web service that enables the communication may be installed. Further, the user terminal 20 may be a smartphone originally owned or a terminal given from a hospital as long as input is performed by the user. Further, a person who performs input to the user terminal 20 is not limited to the user himself/herself, and it may be a terminal that is used by the user's family or a caregiver who cares the user, or a representative who represents the user.

The doctor terminal 30 is, for example, a terminal used by a doctor working in a medical institution such as a hospital to grasp a situation of a user, and executes information processing with the information processing device 10 or the user terminal 20 via the network (NW). The doctor terminal 30 may be, for example, a general-purpose computer such as a workstation or a personal computer, or a portable communication device or the like such as a smartphone. In such a doctor terminal 30, an application or the like capable of communicating with the information processing device 10 or the user terminal 20 may be installed, or a browser for accessing a web service that enables the communication may be installed.

### Hardware Structure

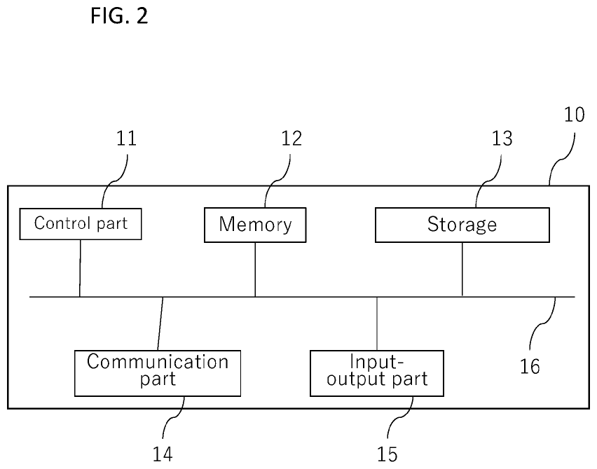

FIG. 2 illustrates an example of a hardware structure of a computer that realizes the information processing device 10 according to the present embodiment. The computer includes at least a control part 11, a memory 12, a storage 13, a communication part 14, an input-output part 15, and the like. These are electrically connected to each other via a bus 16.

The control part 11 is an arithmetic device that controls operation of the entire information processing device 10, and performs information processing and the like necessary for control of transmission and reception of data between the elements and execution and authentication processing of applications. For example, the control part 11 is a processor such as a CPU (Central Processing Unit), and executes information processing by executing a program or the like stored in the storage 13 and expanded in the memory 12.

The memory 12 includes a main memory formed of a volatile storage device such as a DRAM (Dynamic Random Access Memory) and an auxiliary memory formed of a non-volatile storage device such as a flash memory or HDD (Hard Disc Drive). The memory 12 is used as a work area or the like of the control part 11 and stores BIOS (Basic Input/Output System) and various setting information that are executed when the information processing device 10 is started.

The storage 13 stores various programs such as application programs. A database storing data used in processing may be built in the storage 13.

The communication part 14 connects the information processing device 10 to a network. The communication part 14 communicates with an external device directly or via a network access point, for example, using a method such as a wired LAN (Local Area Network), a wireless LAN, Wi-Fi (Wireless Fidelity, registered trademark), infrared communication, Bluetooth (registered trademark), short-range or non-contact communication.

The input-output part 15 is, for example, an information input device such as a keyboard, a mouse, and a touch panel, and an output device such as a display.

The bus 16 is commonly connected to the above-described elements and, for example, transmits address signals, data signals, and various control signals.

In the present embodiment, hardware structures of terminals such as computers or smartphones that realize the user terminal 20 and the doctor terminal 30 are the same as the example of the hardware structure of the information processing device 10 illustrated in FIG. 2, and thus, description thereof is omitted.

### Software Structure

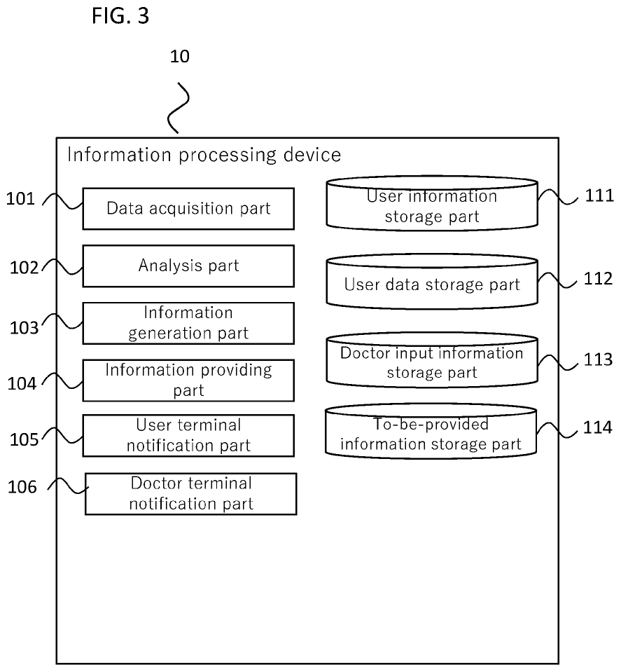

FIG. 3 illustrates an example of a software structure of the information processing device 10 according to the present embodiment. The information processing device 10 can include functional parts including a data acquisition part 101, an analysis part 102, an information generation part 103, an information providing part 104, a user terminal notification part 105 and a doctor terminal notification part 106, and storage parts including a user information storage part 111, a user data storage part 112, a doctor input information storage part 113 and a to-be-provided information storage part 114.

The data acquisition part 101, the analysis part 102, the information generation part 103, the information providing part 104, the user terminal notification part 105 and the doctor terminal notification part 106 are realized by the control part 11 provided in the information processing device 10 by reading out the program stored in the storage 13 to the memory 12 and executing the program. The user information storage part 111, the user data storage part 112, the doctor input information storage part 113 and the to-be-provided information storage part 114 are each realized as a part of a storage area provided by at least one of the memory 12 and the storage 13.

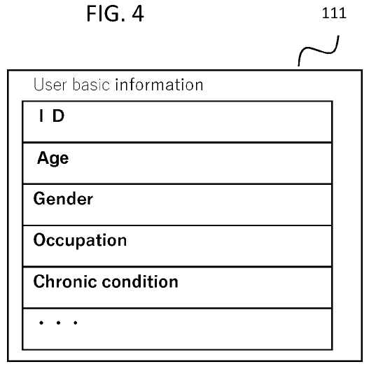

The user information storage part 111 stores, for example, user basic information acquired by the data acquisition part 101. FIG. 4 is an example of a structure of the user basic information stored in the user information storage part 111. The user basic information may be associated with a user ID. The user basic information may include attribute-related information such as user ID, user age, gender, occupation, and place of birth, lifestyle-related information such as chronic condition, medical history, allergies, constitution (obesity, weakness, and the like), eating habits, drinking, smoking, and exercise habits, and the like, and may include user's name, address, height, and weight, when necessary.

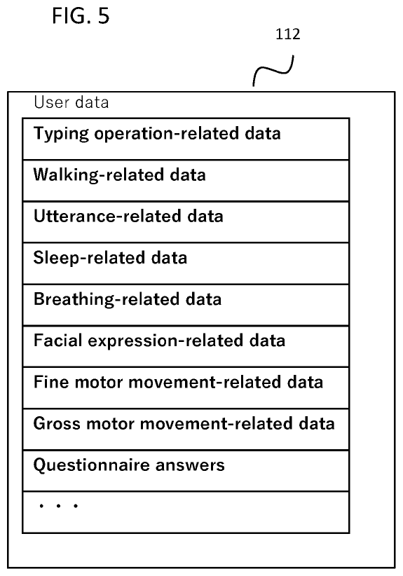

The user data storage part 112 stores user data acquired multiple times by the data acquisition part 101 in a predetermined period. The user data are one or more data related to a direct or indirect motor nervous system dysfunction, and includes, for example, one or more neuromuscular disease-related user data selected from the following (a)-(j), and these are obtained, for example, from the user terminal 20.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices FIG. 5 is an example of a structure of the user data stored in the user data storage part 112. Neuromuscular disease-related user data selected from the above (a)-(j) may be associated with a user ID.
(a) Examples of the “typing operation-related data” include typing speed, accuracy, time and amount. The typing operation-related data is, for example, data of an input operation to the user terminal 20, time required for or speed of an input operation, a re-enter rate, where it is pressed during an input operation, and what words are searched on LINE or in a browser.

The typing operation-related data may be data or the like automatically acquired from GPS, an accelerometer, a text log, screen event data, and the like, which are built in the user terminal 20, or may be data actively acquired from tasks for investigation. The typing operation-related data is, for example, user-typed keystroke data as disclosed in US Patent Application Publication No. 2021/0236044.
(b) Examples of the “walking-related data” include number of steps, walking speed, foot swing angle, ankle movement angle, stride length, arm swing, foot swing, lateral swing of the whole body and rate of falls during walking. The walking-related data are data acquired from a wearable terminal or a smartphone, and is data automatically acquired from an accelerometer built in a wearable terminal. The walking-related data is, for example, walking-related data over a predetermined period detected and recorded using a pedometer built in a wearable terminal or a smartphone, as disclosed in U.S. Pat. No. 9,480,560. The walking-related data may be an image or a video of the user taken with a camera or the like. To easily acquire data without using a complicated measuring equipment and more easily catch signs of a neuromuscular disease through activities of daily living, it is preferable to include step count data as data related to the above (b).
(c) Examples of the “utterance-related data” include voice data of conversation, call record, speaking speed, speaking time, sustained vocalization, number of words, language disorder, frequency of obscure language, pause period, non-speech sound (such as nasal sound) and cough frequency. The utterance-related data include data of continuously acquiring voice recording and evaluating voice deterioration over time. These data are acquired, for example, using a smartphone, a smart watch, a wearable sensor, a computing device, a headset, a headband, or a voice recording device that is a combination of these, as disclosed in International Publication No. 2021/150989. To more easily catch signs of a neuromuscular disease through daily conversations or short audio recordings or the like, it is preferable to include pause period data as data related to the above (c).
(d) Examples of the “sleep-related data” include sleep time, sleep efficiency, eyeball movements, and frequency of awakening. The sleep-related data is, for example, data of circadian rhythm of sleep, sleep onset time and wake up time, and duration acquired using a wearable electronic device, as disclosed in International Publication No. 2019/106230. The sleep-related data may be data such as sleep pattern, sleeping time, wake-up time, sleep depth, and number of REM sleeps acquired using a smartphone, a smart watch, a wearable sensor, or the like, or may be an image or a video of the user taken with a camera or the like.
(e) Examples of the “breathing-related data” include lung function-related data such as vital capacity, forced vital capacity, dyspnea, orthopnea, respiratory failure, and frequency of coughing. The breathing-related data are acquired, for example, using a monitor device (such as a smartphone spirometer), or a spirometer (lung function test). Data acquisition is performed by taking a mouthpiece in the mouth, pinching the nose, breathing according to the voice of a technician, and measuring amounts and speeds of air entering and leaving the lungs.
(f) Examples of the “facial expression-related data” include opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement. The facial expression-related data are acquired, for example, by analyzing facial expressions of a person appearing in a video prepared by a user with emotion recognition AI and outputting the emotions of the person that can be read from the video as numerical data, as disclosed in Japanese Translation of PCT International Application Publication No. 2020-537579. Further, as disclosed in Japanese Patent Application Laid-Open Publication No. 2018-007792, data is acquired from a facial image of a person shown in an image. The facial expression-related data may be an image or a video of the user taken with a camera or the like.
(g) “Fine motor movements” generally refer to movements required for fine and precise operations using hands or fingers, and include writing characters, using chopsticks, fastening buttons, grabbing small objects, and the like. In the present embodiment, the “fine motor movement-related data” is, for example, data obtained from a test in which a drawing test, which is usually performed in analog, is performed on a digital device. Examples of specific tests include tests of drawing a picture on a screen surface of a smartphone, tracing on a presented painting, and moving a geometrical shape from right to left. Signs can be found from accuracy of responses in the tests or times required for the tests. The fine motor movement-related data is acquired, for example, using a technology of performing input to a mobile device with a finger as in a drawing test described in Japanese Patent Application Laid-Open Publication No. 2021-77412. To more easily catch signs of a neuromuscular disease through activities of daily living using a digital device, it is preferable to include data related to fine motor movements performed using the thumb and forefinger as data related to the above (g).
(h) “Gross motor movements” refer to movements that use the whole body such as posture maintenance and locomotion movement, and include walking, running, jumping, throwing things, and the like. In the present embodiment, examples of the “gross motor movement-related data” include movement of changing positions of the arms, going up and down stairs, standing up from a sitting position, and frequency of leg spasms. Here, as the gross motor movement-related data, data acquired by measuring body movements in three dimensions using a camera or sensor as in motion capture, or data of movements of body parts detected using a video of the body can be used. It is performed by capturing body movements. The gross motor movement-related data may be, for example, self-reported information input using a smartphone application, and, for example, limb symptom scale data of the Modified Norris Scale can be used.
(i) The questionnaire answers regarding disease symptoms are, for example, data that can be acquired by having a user or a third party other than the user to input the data to the user terminal 20. The questionnaire answers regarding disease symptoms may be, for example, self-reported information input using a smartphone application. Examples of such questionnaires (surveys) include ALSFRS-R Questionnaire, Rasch Overall ALS Disability Scale (ROADS), CPIB Questionnaire, Neurological Fatigue Index-Motor Neuron Disease (NFIMND), ALS Depression inventory (ADI-12) Questionnaire, ALS Quality of Life Survey (QoL), Survey on demographic and phone usage info, ALS CBS (ALS Cognitive Behavioral Screen), and the like.
(j) The “information automatically collected with built-in sensors of devices” is, for example, data automatically acquired from sensors or applications installed in the user terminal 20. More specifically, these are data automatically acquired from GPS, an accelerometer, call and text logs, screen event data, and the like. Such data is, for example, as disclosed in JMIR Ment Health, 2016 April-June; 3 (2): e16, the above-described (b) “walking-related data” acquired using smartphone applications from GPS and an accelerometer built in a smartphone. To more effectively detect signs of a disease or an abnormal motor function while easily acquiring data with a device carried in daily life or a wearable device, it is preferable to include data automatically acquired from GPS or an accelerometer as data related to the above (j).

In the above, the user data of (a)-(i) may be self-reported information directly input to the user terminal 20 by the user using the user terminal 20. The self-reported information may be information input by a third party other than the user. Further, the self-reported information may be input at a hospital or a place where examination is performed, or may be a compilation of questionnaire answers. The self-reported information may be automatically acquired from the user terminal 20. In addition to the above (a)-(i), the self-reported information may include height and weight of the user.

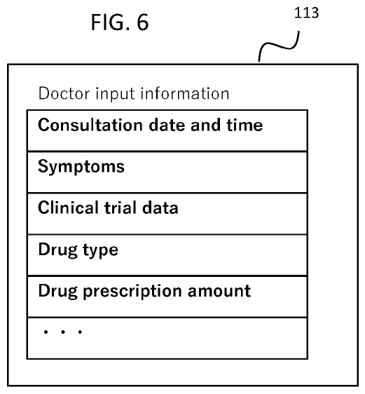

The doctor input information storage part 113 stores doctor input information that is acquired multiple times from the doctor terminal 30 in a predetermined period by the data acquisition part 101, and/or data that is automatically acquired from the doctor terminal 30. FIG. 6 is an example of a structure of the doctor input information stored in the doctor input information storage part 113. Examples of the doctor input information include user consultation information, that is, user's consultation date and time or (k) the “data from medical institutions,” which is one of the user's neuromuscular disease-related user data described above. The doctor input information may be associated with a user ID.
(k) The “data from medical institutions” is medical data that cannot be acquired from the user terminal 20, or data such as medical history. These may be used independently as user data, and can also be used in combination with the above-described user data of (a)-(j) in order to improve accuracy of the to-be-provided information. More specifically, the data from medical institutions is, for example, information acquired from a clinical trial information database (not illustrated in the drawings) that stores data (clinical trial data) acquired in clinical trials conducted at medical institutions or the like. In the case of information acquired from a clinical trial information database, user data may include a date on which a clinical trial was conducted or a data acquisition date. Further, the data from medical institutions may be information acquired from the doctor input information storage part 113 that stores information that is input using the doctor terminal 30 by the doctor who examined the user. Further, the data from medical institutions may be a type or a prescription amount of a drug administered to the user acquired from the doctor input information storage part 113, and may include an administration period of the drug. Further, an image or a video of the patient (user) taken with a camera or the like, or the height and weight of the user may be included.

One or more of the above-described user data of (a)-(k) is data related to a direct or indirect motor nervous system dysfunction, and may be, for example, data related to a motor function included in the ALS function evaluation scale (ALSFRS-R).

Most ALS patients are seen with random asymmetry symptoms including hand or foot spasms, and muscle weakness and muscle atrophy. Muscle weakness progresses to the forearms, shoulders and lower limbs. Shortly afterwards, fasciculation, spasticity, deep tendon reflex hyperactivity, extensor plantar response, dexterity movement disorder, stiff movements, weight loss, fatigue and difficulty in controlling facial expressions and tongue movements occur. Other symptoms include hoarseness, dysphagia, language ambiguity, and the like, and due to difficulty in swallowing, saliva seems to increase, and the patient is more likely to choke on liquids. In a later stage of the illness, pseudobulbar affect occurs, with inappropriate, involuntary and uncontrollable excessive laughter or crying. The sensory system, consciousness, cognition, spontaneous eye movements, sexual function, and urethral and anal sphincter muscles are usually preserved. Therefore, by utilizing data related to motor functions included in ALSFRS-R, the data can be used as the user's neuromuscular disease-related user data.

Here, “ALSFRS-R” (ALS Functional Rating Scale-Revised) accesses activities of daily living of ALS patients, and includes a total of 12 evaluation items regarding motor dysfunction of limbs, bulbar dysfunction, and respiratory dysfunction. Each item is scored in 5 stages from 0 to 4, and is used in evaluating an overall severity and pathological progression of ALS patients. The 12 items to be evaluated include, for example, (1) speech, (2) salivation, (3) swallowing, (4) handwriting, (5) cutting food and handling utensils, (6) dressing and hygiene, (7) turning in bed and adjusting bed clothes, (8) walking, (9) climbing stairs, (10) dyspnea, (11) orthopnea, and (12) respiratory insufficiency. In addition to ALSFRS-R, examples of ALS evaluation methods include 40-item Amyotrophic Lateral Sclerosis (ALS) Assessment Questionnaire (ALSAQ-40), Japanese ALS Severity Classification, Modified Norris Scale, and the like. By using these items as user data, signs of a neuromuscular disease can be easily grasped.

The above-described user data of (a)-(k) can be used in combination as appropriate. The user data may be updated based on the self-reported information. Further, the self-reported information may be updated each time self-reported information is obtained.

In an embodiment, an information processing device or a program is preferably structured to include and acquire one or more selected from the above (a), (b), (c), (f), (g), (h) and (j) as the user data related to the above (a)-(k). Most of these user data are related to direct motor nervous system dysfunction. Therefore, by acquiring such user data, it is possible to easily detect signs of a disease earlier, even before the signs are perceived by the user himself/herself or a third party.

In an embodiment, an information processing device or a program is preferably structured to acquire at least one or more selected from (c) utterance-related data and (g) fine motor movement-related data as the user data related to the above (a)-(k). These data have a high level of validation and are likely to be highly reliable data. Therefore, by acquiring one or more of these data, it is possible to easily detect signs of a disease with high reliability based on highly reliable data.

Further, in an embodiment, an information processing device or a program is more preferably structured to include and acquire one or more data classified in Group 1 shown below among the user data related to the above (a)-(k). Instead of or in addition to this, it is also more preferably structured to include and acquire one or more data classified in Group 2 shown below. In particular, by combining and acquiring data of the groups, motor nervous system-related data at multiple sites can be acquired. As a result, it is possible to easily detect signs of a disease earlier and with higher accuracy. As in this embodiment, when user data of multiple categories is acquired, for example, only data classified in Group 1 and/or Group 2 may be acquired, or one or more user data other than the data classified in Group 1 and Group 2 may be additionally acquired.

Group 1: One or more selected from (c) utterance-related data and (f) facial expression-related data; and preferably, (c) utterance-related data is at least included.

Group 2: One or more selected from (b) walking-related data, (h) gross motor movement-related data, and (j) information automatically collected with built-in sensors of devices; and preferably, (b) walking-related data is at least included.

Further, in an embodiment, an information processing device or a program is more preferably structured to include and acquire one or more user data classified in Group 3 shown below, in addition to the data of Group 1 and/or Group 2 described above, among the user data related to the above (a)-(k).

Group 3: One or more selected from (a) typing operation-related data and (g) fine motor movement-related data; and preferably, (g) fine motor movement-related data is at least included.

It is considered that the data of Group 1 mainly corresponds to bulbar-governed motor functions (such as facial movements), the data of Group 2 mainly corresponds to lower limb motor functions, and the data of Group 3 mainly corresponds to upper limb motor functions. Therefore, by combining and acquiring these user data, data corresponding to motor functions of the whole body can be comprehensively acquired. As a result, even when signs of a disease appear at specific sites, it is possible to easily detect the signs of the disease earlier and with higher accuracy. Further, by combining these data, even for onset or disease progression that does not appear in evaluation scores of ALSFRS-R or the like, signs of a disease can be detected earlier. As a result, quality of life of the user can be improved.

Examples of preferred combinations of the user data related to the above (a)-(k) include, but are not limited to, the following (I)-(V). In any case, it is possible to easily detect signs of a disease earlier with higher accuracy.
(I): A combination including (c) utterance-related data, (b) walking-related data, and (g) fine motor movement-related data.
(II): A combination including (c) utterance-related data, (h) gross motor movement-related data, and (g) fine motor movement-related data.
(III): A combination including (c) utterance-related data, (b) walking-related data, and (a) typing operation-related data.
(IV): A combination including (f) facial expression-related data, (b) walking-related data, and (g) fine motor movement-related data.
(V): A combination including (c) utterance-related data, (j) information automatically collected with built-in sensors of devices, and (a) typing operation-related data.

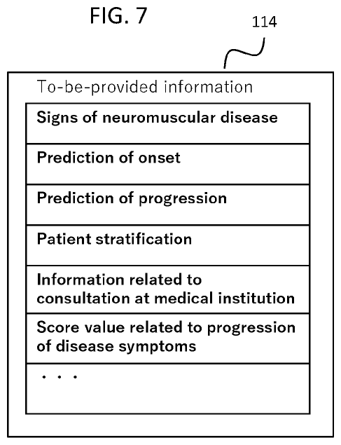

The to-be-provided information storage part 114 stores to-be-provided information generated based on the user data stored in the user information storage part 111 and the user data stored in the user data storage part 112. FIG. 7 is an example of a structure of the to-be-provided information stored in the to-be-provided information storage part 114. Examples of the to-be-provided information include signs of a neuromuscular disease, prediction of onset, prediction of progression, patient stratification, information related to consultation at a medical institution, a score value related to progression of disease symptoms, and the like. An example of the score value related to progression of disease symptoms is a score value used in a case where a fluctuation amount of typing operations obtained by an analysis of the analysis part 102 to be described later is digitized and a value equal to or higher than a threshold is determined as a disease score.

The data acquisition part 101 acquires one or more neuromuscular disease-related user data selected from (a)-(k) multiple times in a predetermined period. By acquiring user data multiple times in a predetermined period, changes in user behavior over time are quantified. The data acquisition part 101 may acquire user data directly from the user terminal 20 or the doctor terminal 30, or may acquire user data via another data server. Information acquired by the data acquisition part 101 is stored in the user information storage part 111, the user data storage part 112 or the doctor input information storage part 113. The data acquisition part 101 may accept, for example, user data input by the user or input by a person other than the user, such as a family member, a friend, a caregiver, or a representative of the user, and may accept data input by two or more people.

The data acquisition part 101 may passively or actively acquire user data. Here, “passively acquired data” is data automatically acquired from GPS, an accelerometer, call and text logs, screen event data, and the like as in (j) described above and refers to data generated without direct involvement of an object person, such as GPS traces and call records. Further, “actively acquired data” is data acquired from tasks (questionnaire answers, input operations using fingers, and the like), and refers to data that requires active participation from an object person for its generation. For example, by utilizing, in a mutually complementary manner, the data passively acquired from sensors such as GPS and accelerometers and logs such as telephone usage logs and communication logs, and the like of the user terminal 20 and the data actively acquired from tasks such as answering questionnaires and inputting with fingers, more highly accurate information can be provided.

The data acquisition part 101 may continuously acquire the above-described user data in a predetermined period. By continuously acquiring the user data, data related to a fluctuation amount of the user data can be acquired. Patients with neuromuscular disease are more likely to get tired and may experience changes in patterns of daily life. Therefore, it is thought that it may be easier to catch signs of a neuromuscular disease by looking at user-specific patterns of sleep, breathing, and the like. Therefore, the more the user data that can be continuously acquired, the better.

The analysis part 102 analyzes signs of a neuromuscular disease from a fluctuation amount of the user data acquired by the data acquisition part 101. That is, by analyzing quality of data obtained from the one or more user data selected from (a)-(k), subtle signs of a neuromuscular disease are caught from results of the analysis. For example, presence or absence of an abnormal value pattern is detected from a fluctuation amount of the user data, and a sign of a dysfunction is predicted from these data using AI, and the like. Signs of a neuromuscular disease are generated as to-be-provided information to be provided to a user or a doctor by the information generation part 103.

The information generation part 103 generates the to-be-provided information to be provided to a predetermined terminal based on the user data acquired by the data acquisition part 101. That is, based on the user data of (a)-(k), that is, by using (a)-(k) independently or in combination, the to-be-provided information to be provided to a user or a doctor is generated. A combination of the user data can be appropriately selected according to intended to-be-provided information or quality of the acquired user data. Further, the information generation part 103 generates information related to signs of a neuromuscular disease as to-be-provided information from analysis results of the analysis part 102. The generated information is transmitted to the user terminal 20 and the doctor terminal 30 by the information providing part 104. The information generated by the information generation part 103 is stored in the to-be-provided information storage part 114.

Examples of the to-be-provided information to be provided to a user include, as described above, not only signs of neuromuscular diseases but also prediction of onset, prediction of progression, patient stratification, information related to consultation at a medical institution, and a score value related to progression of disease symptoms. By providing these to-be-provided information, the user can receive an early consultation, and the doctor can perform an accurate diagnosis and consider care according to a difference in site of onset.

The information providing part 104 provides the to-be-provided information generated by the information generation part 103 to a predetermined terminal used by either the user, the user's family, or the doctor. The terminal to which the information providing part 104 provides information may be the user terminal 20 used by the user, may be the doctor terminal 30, and may be another terminal used by a third party such as an insurance company, a pharmaceutical company, a patient group, a research institution, or a financial institution.

The user terminal notification part 105 notifies, for example, the user of a message prompting the user to acquire user data at a preset timing. The timing may be set for each user, and, for example, an interval or a time slot can be set such that notification is performed at a predetermined time every day, or notification is performed once a week, or the like. Further, a warning may be issued based on the to-be-provided information notified from the information providing part 104.

For example, when to-be-provided information of a user is acquired from the information providing part 104, the doctor terminal notification part 106 notifies, at a preset timing, a message prompting confirmation of the to-be-provided information. Further, for example, when a reservation for consultation is input from the user terminal 20 and the reservation information is acquired via the information processing device 10, notification of the reservation information may be performed.

### Flow of Information Processing

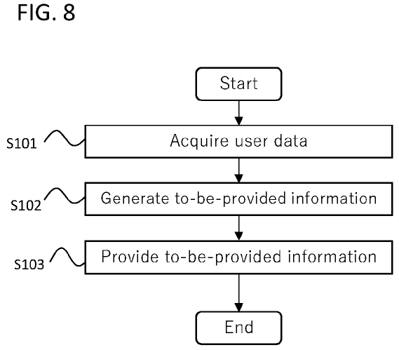

FIG. 8 illustrates a flow of processing executed in the information processing device 10 according to the present embodiment.

First, as preprocessing of the present processing, the data acquisition part 101 of the information processing device 10 accepts information input about the user and stores user basic information in the user information storage part 111. When the user basic information is already stored in the user information storage part 111, by accepting inputs of a user ID and the like, the user basic information required for information processing according to the present embodiment can also be referenced.

Next, the data acquisition part 101 acquires one or more neuromuscular disease-related user data selected from (a)-(k) described above (S101). These data may be acquired by performing an operation for acquiring data held by the user terminal 20, the doctor terminal 30, or another server, or may be automatically acquired by an installed application.

The information generation part 103 generates to-be-provided information to be provided to a predetermined terminal based on the user data acquired by the data acquisition part 101 (S102). That is, based on one or more user data selected from (a)-(k), information related to signs of a neuromuscular disease to be provided to a user, a doctor, or the like is generated.

The information providing part 104 provides the to-be-provided information generated by the information generation part 103 to a predetermined terminal (S103). The terminal to which the information providing part 104 provides the information may be the user terminal 20 used by the user, may be the doctor terminal 30, or may be another terminal of a third party.

In this way, in the information processing device 10 of the present embodiment, neuromuscular disease-related user data is acquired multiple times in a predetermined period, changes in behavior over time are quantified, to-be-provided information to be provided to a predetermined terminal is generated based on the user data, and the result is provided to the predetermined terminal. As a result, signs of a neuromuscular disease of the user can be easily and early detected. As a result, a neuromuscular disease can be detected at an early stage, an appropriate treatment can be provided to the user, and progression of the disease can be prevented. Further, when user data is acquired via a digital device such as a wearable or a smartphone, the user data can be used continuously, non-invasively, and easily as a digital biomarker, and signs of a neuromuscular disease can be easily caught.

The present embodiment has been described above. However, the above-described embodiment is for facilitating understanding of the present invention and is not to be construed as limiting the present invention. The present invention can be modified or improved without departing from its spirit, and the present invention also includes its equivalent. For example, in addition to the information processing device, the present specification also discloses embodiments related to an information processing system, an information processing method using a computer, and a program for causing a computer to execute the information processing method. For points that are not particularly described in relation to these contents, each of the embodiments described in the present specification can be independently adopted or two or more of the embodiments can be adopted in combination as appropriate.

For example, in the present embodiment, for convenience of description, one information processing device 10, one user terminal 20 and one doctor terminal 30 are illustrated. However, it is also possible that, with respect to the information processing device 10, multiple user terminals 20 or multiple doctor terminals 30 are connected via the network (NW). Further, the information processing device 10 is assumed to be one computer. However, without being limited to this, it is also possible that a system is formed by distributing functional parts and storage parts in multiple computers. For example, it is also possible that the storage parts of the information processing device 10 are provided in a database server, and the information processing device 10 accesses the database server. Further, the functional parts can be distributed and provided in multiple computers.

Further, it is also possible that the information processing device 10 is a user terminal used by a user and is structured to access a separately provided database server.

Further, a structure other than the functional parts and structural parts included in FIG. 3 may be included. Further, steps other than the steps included in FIG. 8 may be added. For example, it is also possible that, in S101, user data is continuously acquired, and a fluctuation amount of the data is analyzed by the analysis part 102, and in S102, the information generation part 103 uses results of the analysis to generate to-be-provided information to be provided to a predetermined terminal.

Further, in S103, or after S103, a warning may be issued to the user terminal 20 based on the to-be-provided information. Further, an evaluation part that performs evaluation of drug efficacy in clinical trials based on the to-be-provided information may be provided, and the doctor terminal 30 may be notified of results of the evaluation. Further, based on the evaluation or the to-be-provided information, it may be set to encourage the user to see a doctor. In this case, an appointment for consultation may be accepted and the doctor terminal 30 may be notified.

As a result of the above evaluation, for a user suspected of having a neuromuscular disease, a method for treating the neuromuscular disease may be provided. For example, a step of administering a neuromuscular disease therapeutic agent may be included. Examples of neuromuscular disease therapeutic agents include: ALS therapeutic agents such as edaravone and rilzole; spinocerebellar degeneration therapeutic agents such as taltirelin and protirelin; Parkinson's disease therapeutic agents such as L-dopa and apomorphin; and the like. Further, through the above evaluation, evaluation (new clinical indicators/patient stratification) of drug efficacy in clinical trials may be performed.

The effects described in the present specification are explanatory or exemplary and are not limited. That is, the technology according to the present disclosure may have other effects apparent to those skilled in the art from the description of the present specification, in addition to the above effects, or in place of the above effects.

In order to catch signs of a neuromuscular disease at an early stage, start appropriate treatment at an early stage, and suppress progression of the disease, there is a need to objectively and easily catch the signs of the neuromuscular disease.

The present invention relates to an information processing device that can objectively and easily catch signs of a neuromuscular disease.

An information processing device according to an embodiment of the present invention includes:
a data acquisition part that acquires one or more neuromuscular disease-related user data selected from the following (a)-(k) multiple times in a predetermined period;
and an information generation part that generates to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

An information processing device according to an embodiment of the present invention is capable of easily catching signs of a neuromuscular disease.

(1) An information processing device according to an embodiment of the present invention includes:
a data acquisition part that acquires one or more neuromuscular disease-related user data selected from the following (a)-(k) multiple times in a predetermined period; and
an information generation part that generates to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

(2) In the information processing device of (1), the data acquisition part may continuously acquire the user data in the predetermined period.

(3) In the information processing device (1) or (2), the data acquisition part may passively or actively acquire the user data.

(4) In the information processing device of (1) to (3), the to-be-provided information may be information related to at least one of signs of a neuromuscular disease, prediction of onset, prediction of progression, patient stratification, information related to consultation at a medical institution, and a score value related to progression of disease symptoms.

(5) The information processing device of (1) to (4) may include an analysis part that analyzes the signs of the neuromuscular disease from a fluctuation amount of the user data, and the information generation part may generate the signs as the to-be-provided information.

(6) In the information processing device of (1) to (5), the user data may include data related a motor function included in the ALS function evaluation scale (ALSFRS-R).

(7) In the information processing device of (1) to (6), the user data may include self-reported information of a user.

(8) In the information processing device of (7), the self-reported information may be acquired from a user terminal used by the user.

(9) The information processing device of (1) to (8) may further include an information providing part, and the information providing part may notify a terminal used by either the user, the user's family, or a doctor of the to-be-provided information.

(10) In the information processing device of (1) to (9), the user data may be data related to a direct or indirect motor nervous system dysfunction.

(11) In the information processing device of (1) to (10), the neuromuscular disease may include amyotrophic lateral sclerosis (ALS).

(12) An information processing device according to another embodiment of the present invention includes:
a data acquisition part that acquires one or more direct or indirect motor nervous system dysfunction-related user data selected from the following (a)-(k) multiple times in a predetermined period; and
an information generation part that generates to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

(13) An information processing system according to yet another embodiment of the present invention includes:
a data acquisition part that acquires one or more neuromuscular disease-related user data selected from the following (a)-(k) multiple times in a predetermined period; and
an information generation part that generates to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

(14) An information processing method, in which a computer is used, according to still another embodiment of the present invention includes:
acquiring one or more neuromuscular disease-related user data selected from the following (a)-(k) multiple times in a predetermined period; and
generating to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

(15) According to still another embodiment of the present invention is a program for causing a computer to execute an information processing method. The program causes the computer to execute as the information processing method including:
acquiring one or more neuromuscular disease-related user data selected from the following (a)-(k) multiple times in a predetermined period; and
generating to-be-provided information to be provided to a predetermined terminal based on the user data.
(a) One or more typing operation-related data selected from typing speed, accuracy, time and amount
(b) One or more walking-related data selected from number of steps, walking speed, foot swing angle, ankle movement angle, stride length, arm swing, foot swing, lateral swing of the whole body and rate of falls during walking
(c) One or more utterance-related data selected from voice data of conversation, call record, speaking speed, speaking time, sustained vocalization, number of words, language disorder, frequency of obscure language, pause period, non-speech sound and cough frequency
(d) One or more sleep-related data selected from sleep time, sleep efficiency, eyeball movements, and frequency of awakening
(e) One or more breathing-related data selected from vital capacity, forced vital capacity, dyspnea, orthopnea, respiratory failure, and frequency of coughing
(f) One or more facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement
(g) One or more fine motor movement-related data selected from user taps, inputs, swipes and draws entered into a digital device
(h) One or more gross motor movement-related data selected from arm position-changing movements, going up and down stairs, standing up from a sitting position, frequency of leg cramps
(i) Questionnaire answers regarding disease symptoms
(j) Information automatically collected with built-in sensors of devices
(k) Data from medical institutions

(16) In the information processing device of (1) to (12), the user data may include one or more data selected from the above (a), (b), (c), (f), (g), (h) and (j).

(17) In the information processing device of (16), the user data may include one or more data selected from the above (c) and (g).

(18) In the information processing device of (16), the user data may include data of the following Group 1.
Group 1: one or more selected from data of the above (c) and (f)

(19) In the information processing device of (18), at least data of the above (c) may be included.

(20) In the information processing device of (16), (18) or (19), the user data may further include data of the following Group 2.
Group 2: one or more selected from data of the above (b), (h) and (j)

(21) In the information processing device of (16) and (18) to (20), the user data may further include data of the following Group 3.
Group 3: one or more selected from data of the above (a) and (g)

(22) In the information processing device of (21), at least data of the above (g) may be included.

(23) In the information processing device of (18) to (22), the user data may include one or more data of each of Group 1, Group 2 and Group 3.

(24) In the information processing device of (16), the user data may include any of the following combinations (I)-(V).
(I): a combination including data of the above (c), (b), and (g)
(II): a combination including data of the above (c), (h), and (g)
(III): a combination including data of the above (c), (b), and (a)
(IV): a combination including data of the above (f), (b), and (g)
(V): a combination including data of the above (c), (j), and (a)

(25) In the information processing system of (13), the user data may include one or more data selected from the above (a), (b), (c), (f), (g), (h) and (j).

(26) In the information processing system of (25), the user data may include one or more data selected from the above (c) and (g).

(27) In the information processing system of (25), the user data may include data of the following Group 1.
Group 1: one or more selected from data of the above (c) and (f)

(28) In the information processing system of (27), at least data of the above (c) may be included.

(29) In the information processing system of (25) and (27) or (28), the user data may further include data of the following Group 2.
Group 2: one or more selected from data of the above (b), (h) and (j)

(30) In the information processing system of (25) and (27) to (29), the user data may further includes data of the following Group 3.
Group 3: one or more selected from data of the above (a) and (g)

(31) In the information processing system of (30), at least data of the above (g) may be included.

(32) In the information processing system of (25) to (31), the user data may include one or more data of each of Group 1, Group 2 and Group 3.

(33) In the information processing system of (25), the user data may include any of the following combinations (I)-(V).
(I): a combination including data of the above (c), (b), and (g)
(II): a combination including data of the above (c), (h), and (g)
(III): a combination including data of the above (c), (b), and (a)
(IV): a combination including data of the above (f), (b), and (g)
(V): a combination including data of the above (c), (j), and (a)

(34) In the information processing method of (14), the user data may include one or more data selected from the above (a), (b), (c), (f), (g), (h) and (j).

(35) In the information processing method of (34), the user data may include one or more data selected from the above (c) and (g).

(36) In the information processing method of (34), the user data may include data of the following Group 1.
Group 1: one or more selected from data of the above (c) and (f)

(37) In the information processing method of (36), at least data of the above (c) may be included.

(38) In the information processing method of (34), (36) or (37), the user data may further include data of the following Group 2.
Group 2: one or more selected from data of the above (b), (h) and (j)

(39) In the information processing method of (34) and (36) to (38), the user data may further include data of the following Group 3.
Group 3: one or more selected from data of the above (a) and (g)

(40) In the information processing method of (34) to (39), the user data may include one or more data of each of Group 1, Group 2 and Group 3.

(41) In the information processing method of (34), the user data may include any of the following combinations (I)-(V).
(I): a combination including data of the above (c), (b), and (g)
(II): a combination including data of the above (c), (h), and (g)
(III): a combination including data of the above (c), (b), and (a)
(IV): a combination including data of the above (f), (b), and (g)
(V): a combination including data of the above (c), (j), and (a)

(42) In the program of (15), the user data may include one or more data selected from the above (a), (b), (c), (f), (g), (h) and (j).

(43) In the program of (42), the user data may include one or more data selected from the above (c) and (g).

(44) In the program of (42), the user data may include data of the following Group 1.
Group 1: one or more selected from data of the above (c) and (f)

(45) In the program of (42), at least data of the above (c) may be included.

(46) In the program of (42), (44) or (45), the user data may further include data of the following Group 2.
Group 2: one or more selected from data of the above (b), (h) and (j)

(47) In the program of (42) and (44) to (46), the user data may further include data of the following Group 3.
Group 3: one or more selected from data of the above (a) and (g)

(48) In the program of (47), at least data of the above (g) may be included.

(49) In the program of (42) to (48), the user data may include one or more data of each of Group 1, Group 2 and Group 3.

(50) In the program of (42), the user data may include any of the following combinations (I)-(V).
(I): a combination including data of the above (c), (b), and (g)
(II): a combination including data of the above (c), (h), and (g)
(III): a combination including data of the above (c), (b), and (a)
(IV): a combination including data of the above (f), (b), and (g)
(V): a combination including data of the above (c), (j), and (a)

Obviously, numerous modifications and variations of the present invention are possible in light of the above teachings. It is therefore to be understood that within the scope of the appended claims, the invention may be practiced otherwise than as specifically described herein.

1: An information processing device, comprising:
circuitry configured to acquire at least one neuromuscular disease-related user data a plurality of times in a predetermined period and generate to-be-provided information to be provided to a predetermined terminal based on the user data,
wherein the at least one neuromuscular disease-related user data includes at least one typing operation-related data selected from typing speed, accuracy, time and amount, at least one walking-related data selected from number of steps, walking speed, foot swing angle, ankle movement angle, stride length, arm swing, foot swing, lateral swing of the whole body and rate of falls during walking, at least one utterance-related data selected from voice data of conversation, call record, speaking speed, speaking time, sustained vocalization, number of words, language disorder, frequency of obscure language, pause period, non-speech sound and cough frequency, at least one sleep-related data selected from sleep time, sleep efficiency, eyeball movements, and frequency of awakening, at least one breathing-related data selected from vital capacity, forced vital capacity, dyspnea, orthopnea, respiratory failure, and frequency of coughing, at least one facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement, at least one fine motor movement-related data selected from user taps, inputs, swipes and draws entered into a digital device, at least one gross motor movement-related data selected from arm position-changing movements, going up and down stairs, standing up from a sitting position, and frequency of leg cramps, questionnaire answers regarding disease symptoms, information automatically collected with built-in sensors of devices, and data from medical institutions.

2: The information processing device according to claim 1, wherein the circuitry is configured to continuously acquire the user data in the predetermined period.

3: The information processing device according to claim 1, wherein the circuitry is configured to passively or actively acquire the user data.

4: The information processing device according to claim 1, wherein the to-be-provided information is information related to at least one of signs of a neuromuscular disease, prediction of onset, prediction of progression, patient stratification, information related to consultation at a medical institution, and a score value related to progression of disease symptoms.

5: The information processing device according to claim 1, wherein the circuitry is configured to analyze the signs of the neuromuscular disease from a fluctuation amount of the user data and generate the signs as the to-be-provided information.

6: The information processing device according to claim 1, wherein the user data includes data related a motor function included in ALS function evaluation scale, ALSFRS-R.

7: The information processing device according to claim 1, wherein the user data includes self-reported information of a user.

8: The information processing device according to claim 7, wherein the self-reported information is acquired from a user terminal used by the user.

9: The information processing device according to claim 1, wherein the circuitry is configured to notify a terminal used by either the user, the user's family, or a doctor of the to-be-provided information.

10: The information processing device according to claim 1, wherein the user data is data related to a direct or indirect motor nervous system dysfunction.

11: The information processing device according to claim 1, wherein the neuromuscular disease includes amyotrophic lateral sclerosis.

12: An information processing device, comprising:
circuitry configured to acquire at least one direct or indirect motor nervous system dysfunction-related user data a plurality of times in a predetermined period and generate to-be-provided information to be provided to a predetermined terminal based on the user data,
wherein at least one direct or indirect motor nervous system dysfunction-related user data includes at least one typing operation-related data selected from typing speed, accuracy, time and amount, at least one walking-related data selected from number of steps, walking speed, foot swing angle, ankle movement angle, stride length, arm swing, foot swing, lateral swing of the whole body and rate of falls during walking, at least one utterance-related data selected from voice data of conversation, call record, speaking speed, speaking time, sustained vocalization, number of words, language disorder, frequency of obscure language, pause period, non-speech sound and cough frequency, at least one sleep-related data selected from sleep time, sleep efficiency, eyeball movements, and frequency of awakening, at least one breathing-related data selected from vital capacity, forced vital capacity, dyspnea, orthopnea, respiratory failure, and frequency of coughing, at least one facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement, at least one fine motor movement-related data selected from user taps, inputs, swipes and draws entered into a digital device, at least one gross motor movement-related data selected from arm position-changing movements, going up and down stairs, standing up from a sitting position, and frequency of leg cramps, questionnaire answers regarding disease symptoms, information automatically collected with built-in sensors of devices, and data from medical institutions.

13: An information processing system, comprising:
circuitry configured to acquire at least one neuromuscular disease-related user data a plurality of times in a predetermined period and generate to-be-provided information to be provided to a predetermined terminal based on the user data,
wherein at least one neuromuscular disease-related user data includes at least one typing operation-related data selected from typing speed, accuracy, time and amount, at least one walking-related data selected from number of steps, walking speed, foot swing angle, ankle movement angle, stride length, arm swing, foot swing, lateral swing of the whole body and rate of falls during walking, at least one utterance-related data selected from voice data of conversation, call record, speaking speed, speaking time, sustained vocalization, number of words, language disorder, frequency of obscure language, pause period, non-speech sound and cough frequency, at least one sleep-related data selected from sleep time, sleep efficiency, eyeball movements, and frequency of awakening, at least one breathing-related data selected from vital capacity, forced vital capacity, dyspnea, orthopnea, respiratory failure, and frequency of coughing, at least one facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement, at least one fine motor movement-related data selected from user taps, inputs, swipes and draws entered into a digital device, at least one gross motor movement-related data selected from arm position-changing movements, going up and down stairs, standing up from a sitting position, and frequency of leg cramps, questionnaire answers regarding disease symptoms, information automatically collected with built-in sensors of devices, and data from medical institutions.

14: An information processing method, comprising:
acquiring at least one neuromuscular disease-related user data a plurality of times in a predetermined period; and
generating to-be-provided information to be provided to a predetermined terminal based on the user data,
wherein the at least one neuromuscular disease-related user data includes at least one typing operation-related data selected from typing speed, accuracy, time and amount, at least one walking-related data selected from number of steps, walking speed, foot swing angle, ankle movement angle, stride length, arm swing, foot swing, lateral swing of the whole body and rate of falls during walking, at least one utterance-related data selected from voice data of conversation, call record, speaking speed, speaking time, sustained vocalization, number of words, language disorder, frequency of obscure language, pause period, non-speech sound and cough frequency, least one sleep-related data selected from sleep time, sleep efficiency, eyeball movements, and frequency of awakening, at least one breathing-related data selected from vital capacity, forced vital capacity, dyspnea, orthopnea respiratory failure, and frequency of coughing, at least one facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement, at least one fine motor movement-related data selected from user taps, inputs, swipes and draws entered into a digital device, at least one gross motor movement-related data selected from arm position-changing movements, going up and down stairs, standing up from a sitting position, and frequency of leg cramps, questionnaire answers regarding disease symptoms, information automatically collected with built-in sensors of devices, and data from medical institutions.

15: A non-transitory computer readable medium including a program stored therein that when executed by a computer, causes the computer to execute an information processing method comprising:
acquiring at least one neuromuscular disease-related user data a plurality of times in a predetermined period; and
generating to-be-provided information to be provided to a predetermined terminal based on the user data,
wherein at least one neuromuscular disease-related user data includes at least one typing operation-related data selected from typing speed, accuracy, time and amount, at least one walking-related data selected from number of steps, walking speed, foot swing angle, ankle movement angle, stride length, arm swing, foot swing, lateral swing of the whole body and rate of falls during walking, at least one utterance-related data selected from voice data of conversation, call record, speaking speed, speaking time, sustained vocalization, number of words, language disorder, frequency of obscure language, pause period, non-speech sound and cough frequency, at least one sleep-related data selected from sleep time, sleep efficiency, eyeball movements, and frequency of awakening, at least one breathing-related data selected from vital capacity, forced vital capacity, dyspnea, orthopnea, respiratory failure, and frequency of coughing, at least one facial expression-related data selected from opening and width between upper and lower lips, lip movement, opening speed and acceleration, spasm, mouth surface, average symmetry ratio of left and right mouth surfaces, vertical positions of eyebrows, eye opening, parallel movement and rotation vector of head tilt, and eyeball movement, at least one fine motor movement-related data selected from user taps, inputs, swipes and draws entered into a digital device, at least one gross motor movement-related data selected from arm position-changing movements, going up and down stairs, standing up from a sitting position, and frequency of leg cramps, questionnaire answers regarding disease symptoms, information automatically collected with built-in sensors of devices, and data from medical institutions.

16: The information processing device according to claim 2, wherein the circuitry is configured to passively or actively acquire the user data.

17: The information processing device according to claim 2, wherein the to-be-provided information is information related to at least one of signs of a neuromuscular disease, prediction of onset, prediction of progression, patient stratification, information related to consultation at a medical institution, and a score value related to progression of disease symptoms.

18: The information processing device according to claim 2, wherein the circuitry is configured to analyze the signs of the neuromuscular disease from a fluctuation amount of the user data and generate the signs as the to-be-provided information.

19: The information processing device according to claim 2, wherein the user data includes data related a motor function included in ALS function evaluation scale, ALSFRS-R.

20: The information processing device according to claim 2, wherein the user data includes self-reported information of a user.

21: The information processing device according to claim 20, wherein the self-reported information is acquired from a user terminal used by the user.

22: The information processing device according to claim 2, wherein the circuitry is configured to notify a terminal used by either the user, the user's family, or a doctor of the to-be-provided information.
